(19) United States
(12) Patent Application Publication
(10) Pub. No.: US 2018/0136238 A1
(43) Pub. Date: May 17, 2018

(54) METHOD TO ACCURATELY CALCULATE THE TRUE TOXICITY OF METALS IN SOILS AND WATER

(71) Applicant: Linsey McLean, Rapid City, SD (US)

(72) Inventor: Linsey McLean, Rapid City, SD (US)

(21) Appl. No.: 15/699,293

(22) Filed: Sep. 8, 2017

Related U.S. Application Data
(60) Provisional application No. 62/385,371, filed on Sep. 9, 2016.

(51) Int. Cl. G01N 33/74; G01N 33/20; G16H 50/30

(52) U.S. Cl. CPC G01N 33/743; G16H 10/40; G16H 50/30; G01N 33/20

(57) ABSTRACT

Currently, the analysis of metals for toxicity evaluation are only assayed for total quantity/quantitative elemental analysis, and no assessment to date evaluates the individual toxicities and sum total toxicities of the individual chemical forms of metals, which can be radically different. A better assessment of true toxicity can be made by looking at the quantitative numbers of each of the chemical forms the metal exists in as a function of one of the different valuations of toxicity, such as LD50 numbers, LD0, LD100, LC50, LOAEL, NOAEL, PEL, REL, TLV, etc., for a new terminology of Toxic Relevance Number (TRN), and the sum of TRN's for the individual chemical forms as TTRN, Total Toxic Relevance Number. While LD 50 is preferred, any of these valuations can be used in the same model to advance our understanding and assessment of true toxicity of a certain metal in a specific exposure.

### TECHNICAL FIELD

The disclosed inventive concept relates to toxicity of soils and water. More particularly, the disclosed inventive concept relates to a method for accurately calculating the true toxicity of metals in soils and water.

### BACKGROUND OF THE INVENTION

The state of technology in chemical analyses today does not include assays for the different chemical forms of metals and minerals in assessing the true toxicity of the exposure being studied. Simply testing for amounts of elemental metals in a given volume or concentration is the only reference we have currently for determining toxicity in exposure to metals in the environment for regulatory purposes. This is a simple elemental analysis, expressed in numerical value per volume.

Qualitative analysis, on the other hand, refers to methods whereby the substance, in this case a metal, is analyzed or identified by the chemical form it exists in, what it is bonded to, the ionic state it exists in, whether organified in an organic form or an inorganic species, the basis of their chemical or physical properties, such as chemical reactivity, solubility, molecular weight, melting point, radiative properties (emission, absorption), mass spectra, nuclear half-life, etc. In effect, the qualitative analysis measures the “qualities” of the metallic compound, which can either increase or decrease toxicity to the living organism. In fact, different chemical forms of metals are well known to have different bioavailabilities of absorption, different tissue targets in the living body, and different toxicities and LD50 numbers.

All metals/minerals have a relationship to each other in Nature. They balance each other. Too much of one will have a negative effect on the other. For good health, they all need to be in proper balance. Toxic metals generated from mining are many, and will compromise many essential minerals for health. When one mineral or metal is too high, it will exert a repressive effect upon its counterpart metal or mineral, causing a deficiency or imbalance. Since nutritional minerals are known to fuel enzyme systems in the body, and the living body is dependent upon enzymes for life itself, since the basal temps of the living body are not high enough to fuel the essential biochemical reactions, compromise of any enzyme system can cause severe health consequences and even death.

(see FIG. 1—MINERAL INTERRELATIONSHIPS WHEEL, in which this “Wheel” identifies the perfect relationship existing between all minerals. The mineral balance must be maintained for proper health. (Source: http://earthwiseagriculture.net/the-mineralwheel/)

Additionally, toxic metals can and do interfere with absorption and metabolism of nutritional essential minerals in the living body, creating physiological nutritional deficiencies, even when the levels of these nutritional minerals are adequate. This then causes a whole different set of health compromises that need to be added to the total toxicity evaluation and assessment. The following chart shows known mineral interactions with the toxic metals associated with mining highlighted. A toxicity of one can cause a deficiency of another. The toxic metals are shown in an overlay below to accurately depict the interference of those toxins on the natural system and their impact to all living things, even plants.

So, to more properly assess the true toxicity of a metal in an environmental exposure, we must know all the chemical forms in which it exists in the sample or environment we are testing, the values of toxicity for all the forms, and the sum total of their toxic expressions.

Here are some of the ways that toxicity can be expressed.

Further, there can be different values for toxicity for exposure and exposure limits for compounds overall:

OSHA: The legal airborne permissible exposure limit (PEL) averaged over an 8-hour work shift.

NIOSH: The recommended airborne exposure limit (REL) which should not be exceeded during any 15-minute work period.

ACGIH: The threshold limit value (TLV) averaged over an 8-hour work shift.

(See FIG. 2—DOSE RESPONSE)

### SUMMARY OF THE INVENTION

The disclosed inventive concept overcomes the problems associated with making accurate determinations of metal toxicity in soils and water. Particularly, the disclosed inventive concept teaches that a better assessment of true toxicity of a metal in an environment can be made by looking at the quantitative assay numbers of each of the chemical forms in which the metal is known to exist as a function of the different valuations of toxicity. Such quantitative assay numbers include LD50 numbers, LD0, LD100, LC50, LOAEL, NOAEL, PEL, REL, TLV, etc., for a new terminology of “Toxic Relevance Number” (TRN), with the summary of TRN's of the individual chemical forms shown as TTRN, “Total Toxic Relevance Number”.

The disclosed inventive concept further provides for the incorporation of evaluation of toxic metals per the Mineral Wheel, by opposition, whereby biochemical interference of chemical forms, tissue targets, uptake, ultimate destination in the living body, etc., gives a more complete addition of toxicity evaluation of toxic metals in exposure and in the environment.

The disclosed inventive concept additionally provides that relevant testing and monitoring of the living body for endocrine disruptors of xeno-estrogens can be done by testing blood for Total Estrogens and Estradiol, as separate assays, not calculated open from another as is commonly done in medical laboratories. The Total Estrogens test is now considered somewhat obsolete, as it is known to pick up other compounds of estrogenic activity. However this is exactly the activity for which we are looking. There are three basic classes of estrogens known in the living body:
Estradiol, which composes approximately 80% of estrogens in the body and is the most potent in activity, made by the ovaries and adrenals
Estrone, which is mostly made and stored in body fat, and the most inflammatory and most associated with environmental exposures
Estriol, which is the only form of estrogen not known to cause cancer, and a breakdown product of the other two, the weakest acting of the estrogens

Setting up a simple math equation using two assayed numbers of Total Estrogens and Estradiol, we can assess a Toxic Body Burden of Estrogen Dominance and Hormone Disruption, as well as monitor the success of detoxification procedures.

$$\frac{Estradiol at 80 \%}{Total estrogens at 100 \%} = \frac{assayed number}{\begin{matrix}Total Estrogens expected and compared \\ with actual assayed number\end{matrix}}$$

### BRIEF DESCRIPTION OF THE DRAWINGS

For a more complete understanding of this invention, reference should now be made to the embodiments illustrated in greater detail in the accompanying drawings and described below by way of examples of the invention wherein:

FIG. 1 is an image of a “Mineral Interrelationships Wheel”;

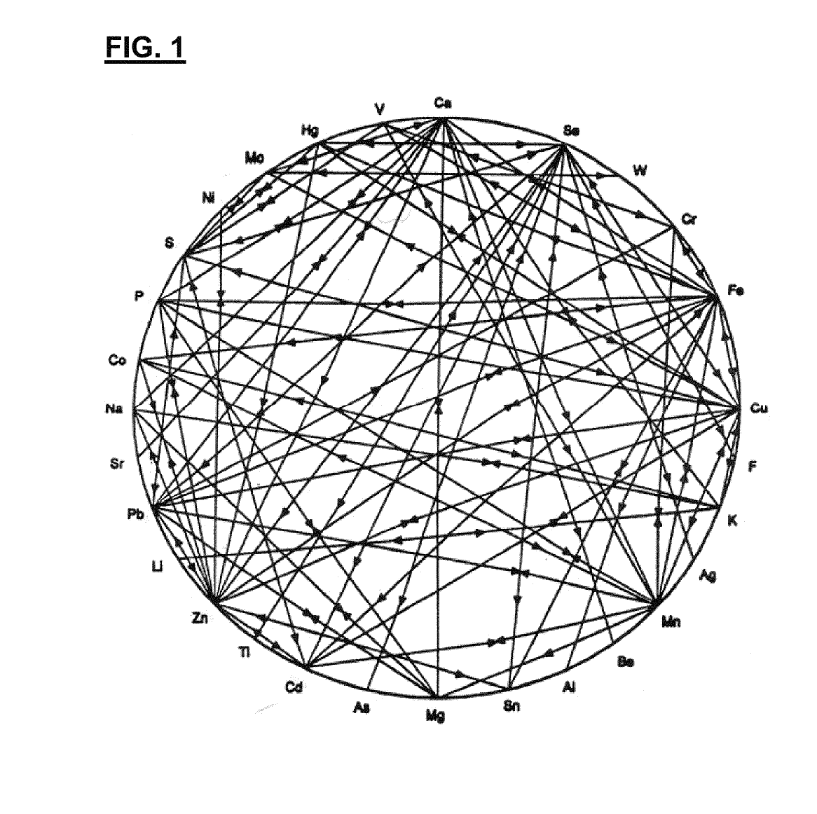

FIG. 2 is a dose/response chart;

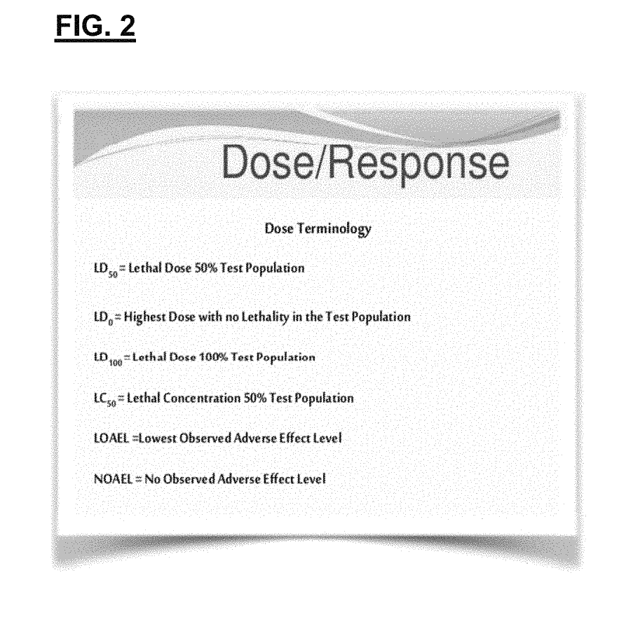

FIG. 3 is a chart showing the values for arsenic compounds;

FIG. 4 illustrates the toxicology of common forms of arsenic;

FIG. 5 is a chart illustrating the phenomenon of bioaccumulation;

FIG. 6 illustrates how inorganic chemical forms become organic chemical forms in nature;

FIG. 7 illustrates the chemical activity of selenium;

FIG. 8 illustrates selenium compounds;

FIG. 9 illustrates the central role of DNA damage and epigenetic defects in DNA repair genes in carcinogenesis;

FIG. 10 illustrates protein pathways; and

FIG. 11 illustrates the insulin chain.

### DETAILED DESCRIPTION OF THE PREFERRED EMBODIMENT

There are many techniques, methods and types of instrumentation used for both quantitative and qualitative assays of metals in polluted soils and waters, and more are being designed all the time. The actual method of assay is not important for our purposes, only the results, as that is what is compared to known LD50 and other referenced numbers.

A better assessment of true toxicity of a metal in an environment can be made by looking at the quantitative assay numbers of each of the chemical forms the metal exists in as a function of one of the different valuations of toxicity, such as LD50 numbers, LD0, LD100, LC50, LOAEL, NOAEL, PEL, REL, TLV etc, for a new terminology of “Toxic Relevance Number” (TRN), with the summary of TRN's of the individual chemical forms shown as TTRN, “Total Toxic Relevance Number”. For purposes of example, we will use just the LD50, however any of these valuations can be used in the same model by multiplying the assayed number by the toxic valuation unit to equal the TRN, with the TRN values of each chemical form added together to equal the TTRN. Now we have a relevant set of numbers that include the differences in toxicity between the different chemical forms of each metal.

Assay value of a specific chemical form×LD50value=TRN(Toxic Relevance Number),LD50

Sum of the TRN numbers of each chemical form of the same metal=TTRN (Total Toxic Relevance Number) for that metal in that particular environment or sample, LD50

### Example

The Animas River Disaster and Arsenic

Arsenic was shown to be 264 ppb for arsenic, above a state limit of 10. This was an elemental analysis only. This number says nothing about the true toxicity expected in living things from exposure to the arsenic levels here.

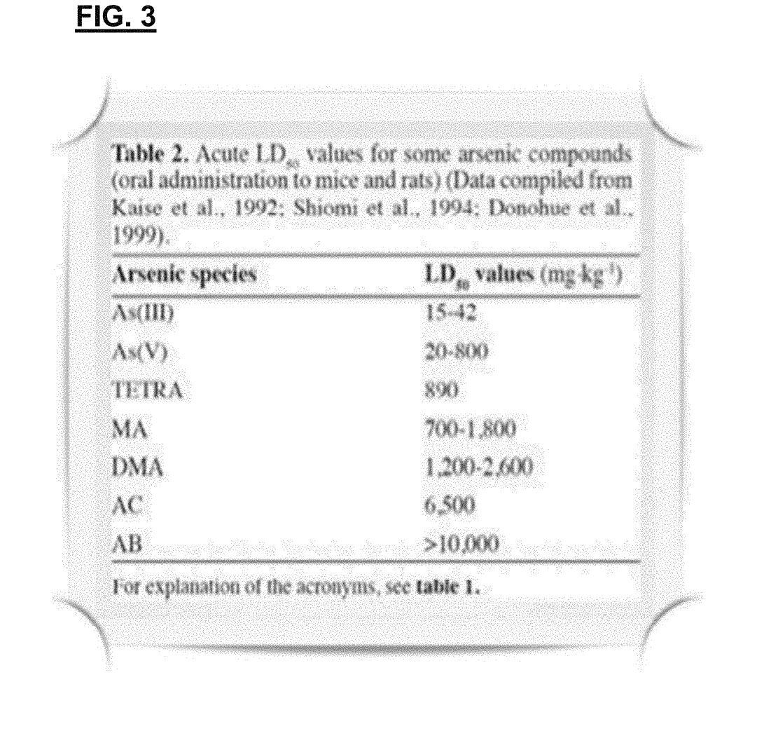

Arsenic occurs in many chemical forms, and each chemical form has a different LD50 rate for toxicity. (See FIG. 3—VALUES FOR ARSENIC COMPOUNDS)

Arsenic is a potent toxicant that may exist in several oxidation states and in a number of inorganic and organic forms, each with its own LD50 values.

The tables below show the drastic differences for LD50 values between different chemical forms of arsenic. Notice that the organified forms of arsenic have a much higher LD50 level, however the situation is quite the opposite for mercury, another toxic metal, where the organified form of methyl mercury and dimethyl mercury are much more toxic than the inorganic forms. For example: the LD50 dose for mercuric sulfide approaches 100,000.0 mg/kg, whereas the organified dimethyl mercury is 0.015 mg./kg, which is orders of magnitude difference. So the TRN and TTRN values would be opposite in number for mercury as compared to arsenic. Another reason for the importance of calculation of the TRN and TTRN values. http://popups.ulg.ac.be/1780-4507/index.php?id=6866

Although the toxicity of organic arsenicals has not been as extensively investigated as inorganic arsenicals, there are sufficient animal data to evaluate the toxicity of methyl arsenates (e.g., monomethylarsonic acid [MMA] and dimethylarsinic acid [DMA]) and roxarsone.

https://www.atsdr.cdc.gov/toxprofiles/tp2-c2.pdf

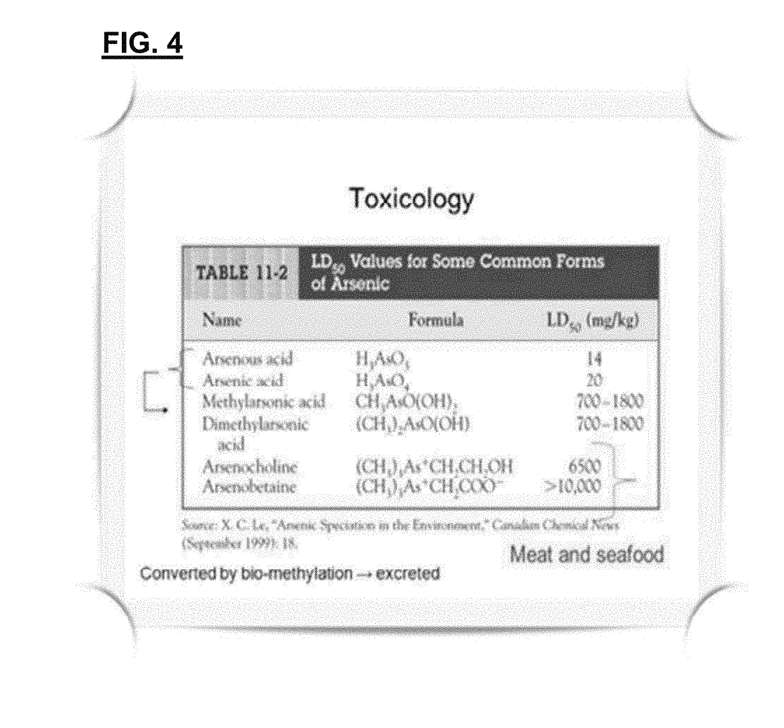

(See FIG. 4—TOXICOLOGY—COMMON FORMS OF ARSENIC)

Relevance of the Method

The Difference Between Inorganic and Organic Compounds:

Organic compounds always contain carbon, while most inorganic compounds do not contain carbon. Also, almost all organic compounds contain carbon-hydrogen or C—H bonds. Organic chemistry is “The Chemistry of Life”.

Metals in an inorganic form have significantly different chemistry in the living body from organically bound minerals. Organic forms of uranium as well as other toxic metals have also been shown to exist and build up in mining areas and mining aquifers, and they are not known to be recoverable by the ion exchange method of ISL recovery, since it is already bound organically and will not bind to the organic synthetic resins.

Reference: Arabian Journal of Chemistry

Volume 4, Issue 4, October 2011, Pages 361-377

Organic forms of any toxic metal are known to be much more long term toxic and much more bioavailable, so that they are able to penetrate the lining of the digestive tract much easier than ionic and inorganic salts that are blocked by their electrical charges. Organic metals have their electrical charges spread over the organic ligand they are bound to, so that they act as a “chelate”, something that the health industry does to minerals to significantly improve absorption of essential minerals, and also make them much more able to enter into direct biochemical reactions in the living body. Organically bound metals under this circumstance, and there is plenty of organic carbon naturally existing with ISL mining aquifer sites as well as sediments in rivers, streams and lakes to make this a complication, will continue to increase in the waste water of the ISL mine as they are not recoverable, adding to the metal burden of the wastewater and also the toxicity of such beyond what would be if the metals remained in an inorganic and ionic or rock form.

All metals have the ability to organify and form organic metallic chelates, where a ligand is bound to the metal, dispersing and spreading its electric charge over the whole molecule. In this form, the living body can and will very efficiently uptake that metallic form through the essentially neutrally charged lining of the gastrointestinal system, where the more highly electric inorganic salt forms of the same mineral are blocked. Thus, organifled forms of all metals are more bioavailable and bioactive. Toxic organified forms are more able to enter and corrupt biochemical metabolic pathways, affecting levels of excretion that would aid in detoxification in case of exposure.

Organification of metals also allows for a higher body tissue level and faster bioaccumulation up the food chain in the environment as well.

Organified toxic metals are most responsible for long term chronic compromises to health and wellness, and even cancer rates. It is well known that organified metals and chelates have different tissue targets and organ destinations in the living body.

Organification of nutritional minerals, as in amino acid chelates, is known for enhanced bioavailability and biometabolism of such nutritional minerals. These organified chelated nutritional forms are very helpful in cases of severe nutritional deficiency, where nutritional status needs to be restored quickly; in genetic compromise where certain nutritional minerals are compromised in absorption, and thereby compromise wellness and lifespan; and also where enhanced bioavailability and absorption are needed to overcome and out-balance an opposing high level toxic metal in the body. (See mineral wheel above) Examples of this “bumper pool” effect would be a person with a high mercury level, given an organified amino acid chelated selenium, in the form of L-selenomethionine, to oppose and bump out the high levels of mercury in the body, restoring health. High levels of cadmium in the body can be addressed by giving zinc amino acid chelate, the natural opposition of cadmium.

### REFERENCE

Twenty five controlled studies by different authors, in five different countries, adverse array of data is presented. These data validate the effectiveness of mineral nutrients presented as amino acid chelates when compared with the ionic forms derived from the inorganic salts. These studies further support the results of numerous laboratory experiments showing increased absorption, assimilation and reduced toxicity of the forms of minerals chelated to amino acids. With little cost and effort animals can be supplemented with amino acid chelates which will promote, with little risk of overdose, a fuller genetic potential achievement as far as mineral requirements are concerned. Results of this supplementation are reflected in increased growth, immunological integrity and more consistent reproduction increased ovulation and conception after first service as a result of increased bioavailability of these nutritional amino acid chelated minerals. Chelated Minerals In Animal Nutrition Rajendran, C. Kathirvelan and V. Balakrishnan, Madras Veterinary College, Chennai, INDIA

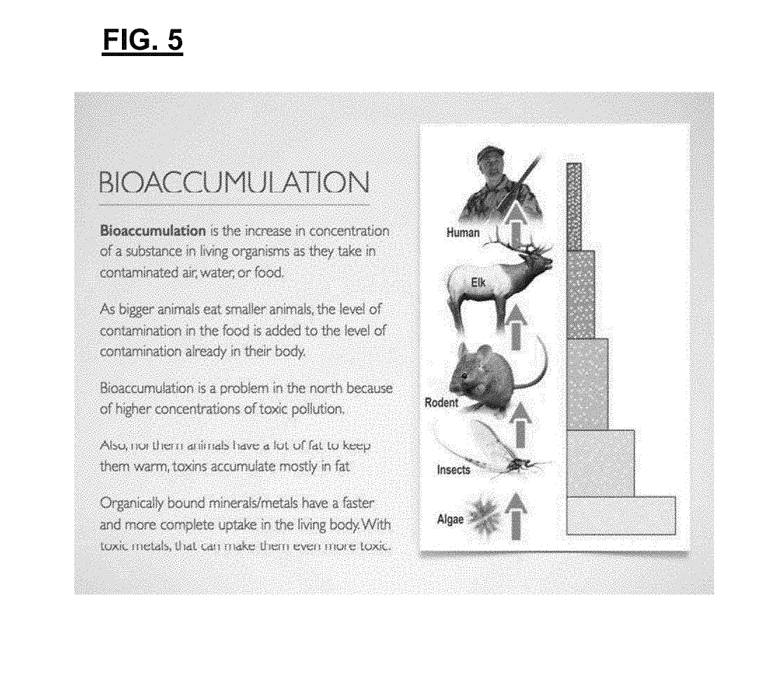

Bloaccumulation of Organifled Toxic Metals Rises Quickly in the Living Systems and the Environment, Rising Up the Food Chain (See FIG. 5—Bioaccumulation)

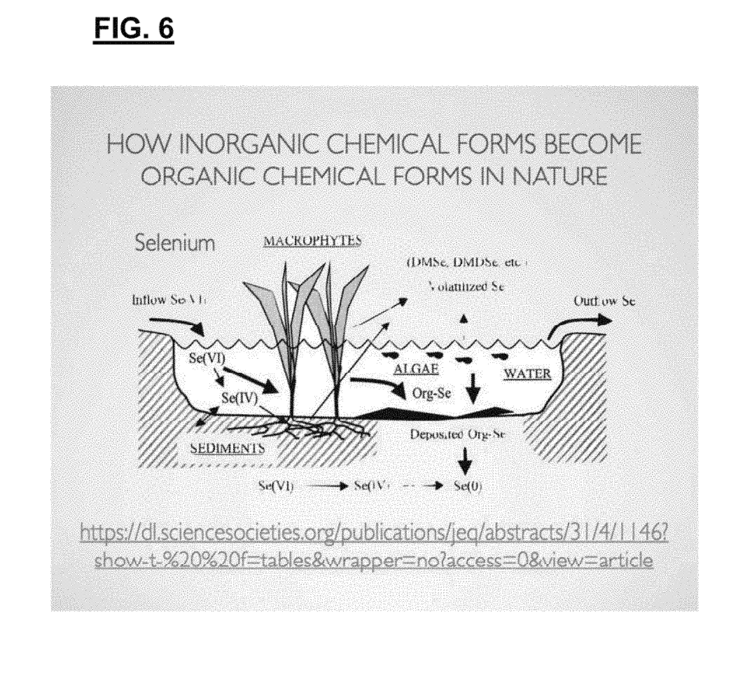

Elemental inorganic forms of metals and minerals are “organified”, bonded with carbon compounds to become organic forms by micro organisms, which are then eaten by simple life forms, which are then eaten by higher animals, and so on, all the way up to man and other top predators at the top of the food chain. As these metals and minerals pass from one body to the next, they are known to concentrate as they move up, with humans and other top predators then suffering the worst consequences from the highest concentration in their tissues and organs. There can be formed many different kinds of organic metal compounds, however, all are not equally bio essential, some are even more toxic as the living body cannot convert them into detox forms. This will depend on which micro organisms are organifying the metals into which compounds. (see FIG. 6—HOW INORGANIC CHEMICAL FORMS BECOME ORGANIC CHEMICAL FORMS IN NATURE)

The Jekyll and Hyde Personalities of Minerals

Even the minerals that we consider necessary for the living body will have different biochemical actions and tissue and organ destinations in the living system. Common case in point: selenium. Selenium is known to have wonderful health effects, preventing cancer, converting the storage form of the storage thyroid hormone T4, to the active form T3 by virtue of fueling an enzyme glutathione peroxidase. This biochemical reaction is absolutely essential to life. Glutathione also doubles as the most powerful antioxidant in the body. Inorganic selenium, on the other hand, is known to cause birth defects of the highest severity. In the inorganic state, selenium, as a consequence of mining, is severely toxic, producing severe deformities. The higher evolved animals above micro organisms are not able to convert quantities of the inorganic forms of minerals, even essential ones like selenium, into the bio compatible organic forms.

When inorganic selenium leaves solidified rock and enters streams, ponds and lake bottoms where micro organisms live, it can and does become organified into different chemical organic forms, only some of which are non toxic and helpful. Others are not.

The figure right, shows how that process happens in Nature. This representation is also true for other toxic metals such as arsenic and mercury, where methyl and dimethyl mercury become far more toxic in the organified forms than their former inorganic elemental form.

Problems with Ion Exchange in Water Purification

“Ion exchange is another method used successfully in industry for the removal of heavy metals from effluent. An ion exchanger is a solid capable of exchanging either cations or anions from the surrounding materials. Commonly used matrices for ion exchange are synthetic organic ion exchange resins. The disadvantage of this method is that it cannot handle concentrated metal solution as the matrix gets easily fouled by organics and other solids in the wastewater. Moreover ion exchange is nonselective and is highly sensitive to the pH of the solution.” (Kurniawan et al., 2006)

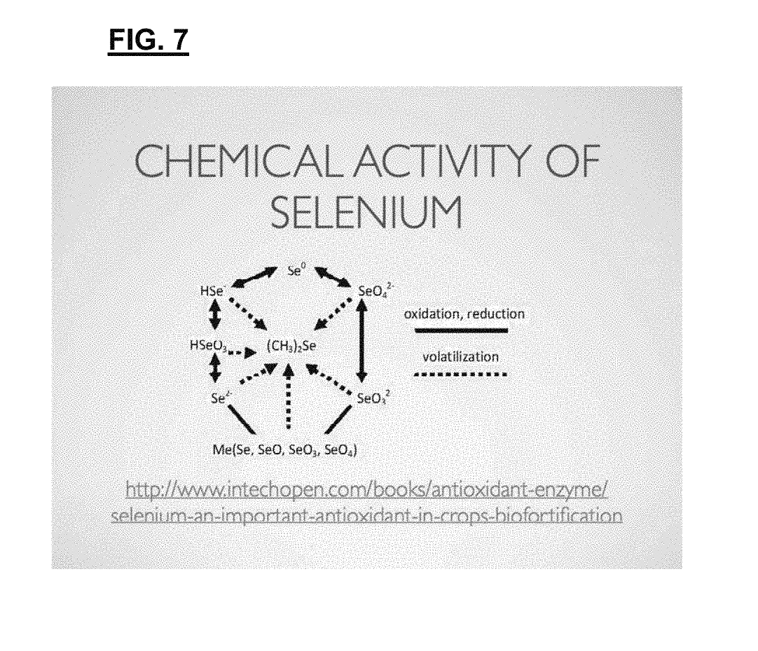

Inorganic salts of metals, most prominent in aquifers, also have different toxicities, and any monitoring of aquifers should include speciations of these different forms so that proper toxicity evaluation can be done. Simply giving the absolute levels of a metal does not tell the whole story. All metallic “salts” are not equal. They can have different solubilities, different melting points, different Ph, different conductivity affecting the central nervous system that relies on electrical signals, and totally different chemistry within the living body. Further, any discussion to the general lay public needs to distinguish between a chemical metallic salt and ordinary table salt, that the public is led to believe will be created as “salt” in a mined aquifer. The figure below shows the many species/chemical forms that selenium, a common metal in mining, can take upon exposure to oxidation/reduction reactions typical within an ISL mining aquifer. Typically, speciation testing, even if monitored by the mining company, is not made available to the public, and is not required by oversight regulatory agencies. (see FIG. 7—CHEMICAL ACTIVITY OF SELENIUM)

Selenium is a poorly regulated toxic metal, and difficult to regulate as far as toxicity and allowable levels are concerned, because of the myriad chemical forms that it can exist in, each with different toxicity. The same can also be said for every other toxic metal as well as nutritional metal. The Jekyll and Hyde personalities of these elements is a very real thing in the natural world. The chart below shows the incongruencies between actual toxicities of some chemical forms of selenium and the regulatory levels allowed.

Most toxicity level charts fail to take into consideration the chemical forms of metals and minerals, which is absolutely critical in assessing any toxicity status. Care for patients suffering from selenium poisoning is usually aimed at treating symptoms. There is no specific antidote or treatments for selenium poisoning.

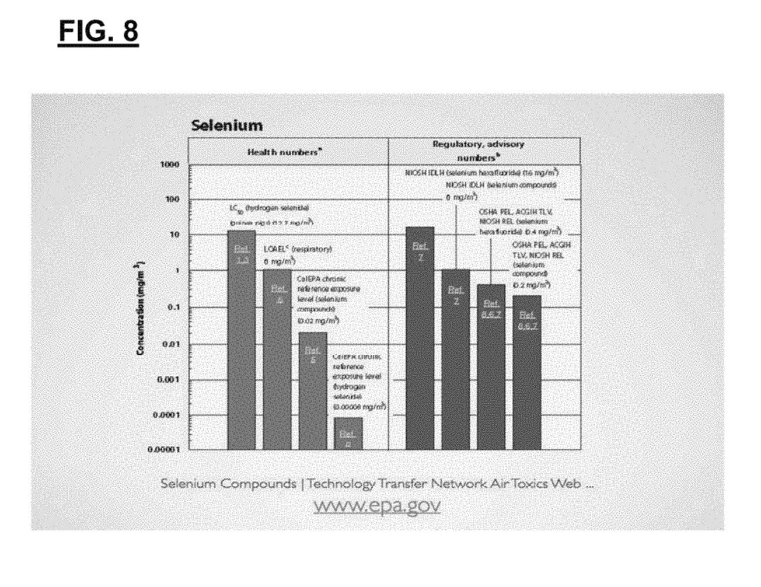

(see FIG. 8—SELENIUM COMPOUNDS)

### REFERENCE

Upper Human Limits for all Minerals and Metals
http://iom.edu/Activies/Nutrition/SummayDRIs/˜/media/Files/Activity%20Files/Nutrition/DRIs/ULs%20for%20Vitamins%20and%20Elements.pdf

Arsenic is Another Major Toxic Metal Pollutant.

Unlike selenium, which has a value in certain chemical forms as a health and life biochemistry promoter, arsenic has not been found to have any health value outside of its use as a parasiticide, and even that use can have toxic consequences.

Arsenic opposes iodine on the mineral wheel of life, and will cause a physiological iodine deficiency by its opposing actions even if there is enough iodine in the diet to counteract general deficiency. Such is the case with all opposing metals and minerals of nutritional minerals. This is how things work in Nature and the living body. Metals like arsenic have their own set of compromising chemistries, but the opposition and interference chemistries of opposing metals and minerals presents a whole new set of pathways for health compromise, independent of the individual roles of the individual metals in actual biochemical reactions. So, by its opposing action on iodine, arsenic can precipitate a whole hypothyroid overlay on the living body, complete with all the health compromises that a hypothyroid body will manifest. High arsenic levels in the living body are completely missed by conventional medicine in assessing treatment for clinical and sub clinical hypothyroid patients. Often, high arsenic levels are found in hair testing of euthyroid/hypothyroid patients in the clinical setting. Increasing iodine supplementation helps this common condition greatly, especially if the iodine supplement is in an amino acid chelated form, for better assimilation and biochemical activity against arsenic in the body.

There is no specific treatment for chronic arsenic poisoning. Once it has been identified further exposure should be avoided. Recovery from the signs and symptoms may take weeks to months from when exposure is stopped. In particular, effects on the nervous system may take months to resolve and in some cases a complete recovery is never achieved.

Epigenetics, a Newly Recognized Toxic Compromise of DNA by Toxic Metals.

Epigenetics is a new study looking at how toxic metals and other environmental toxins can and do affect the gene expression of DNA to cause potentially serious ill health compromises, even death. DNA is actually a set of switches which are found to be controlled by chemical signals from the cell membrane of each cell, which are generated in response to the cell membrane's sensing of the environmental characteristics in the fluid surrounding it. Every living cell is actually floating in a body fluid called lymph. If the cell membrane senses that something is wrong, it sends a chemical signal to the cell nucleus and DNA there to adjust, by turning “on” or “off” certain genetic switches, called “up regulating”, or “down regulating”. This is the living body's way of adapting to its surroundings for survival. This is evolution in progress.

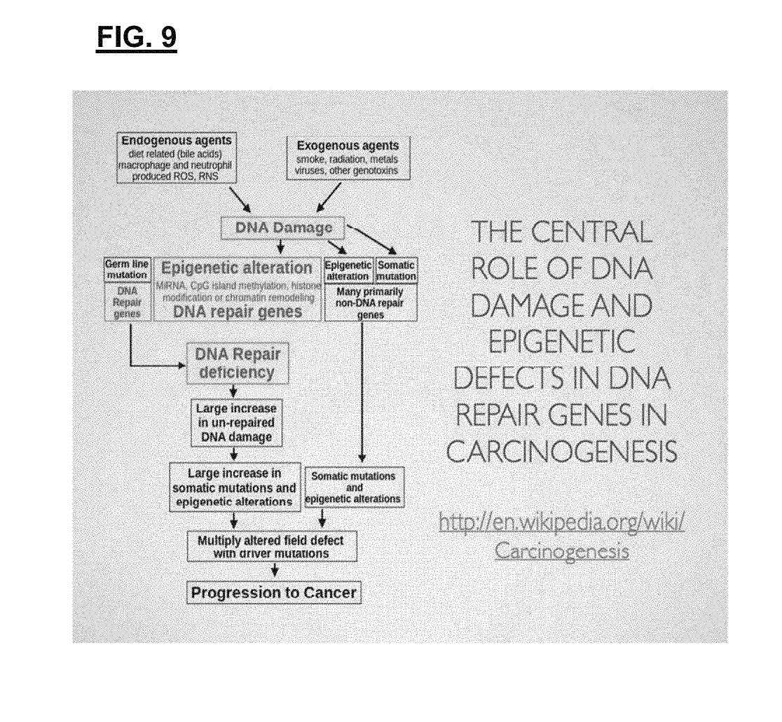

(see FIG. 9—THE CENTRAL ROLE OF DNA DAMAGE AND EPIGENETIC DEFECTS IN DNA REPAIR GENES IN CARCINOGENESIS)

Toxic metals have been found to both “up regulate” and “down regulate” DNA switches, and these switches tripped by epigenetic toxins can remain tripped in up to 5 generations hence, even if the original cause or toxin has been removed in the first generation. The implications for health and humanity for future generations considering epigenetics is mind blowing. The figure above tells the story of epigenetics and the impact on DNA expression, all the way to cancer.

Inorganic forms of minerals, especially selenium and uranium, as well as other heavy metals, which consistently test high in aquifers post mining, have shown to be toxic to living systems of plants, animals and humans in very low levels. Uranium toxicity at low levels has been shown in population statistics of exposed populations such as Native Americans on contaminated and exposed reservations, downwind and downriver from old exposed uranium mines, to be more predisposed to chronic conditions such as: metabolic syndromes, diabetes, behavior and sleep problems, obesity and heart disease, fertility, and morbidity and mortality compromises. These are non-radiological effects of uranium discussed, in that uranium as a metal, actively incorporates itself into the biochemistry of the body. The radiological effects are another subject, not involving the actual chemical reactions such are described here.

### REFERENCE

Heavy Metal Uranium Affects the Brain Cholinergic System in Rat Following Subchronic and Chronic Exposure

“Previous studies have shown that uranium is present in the brain and alters behavior, notably locomotor activity, sensorimotor ability, sleep/wake cycle and the memory process, but also metabolism of neurotransmitters. The cholinergic system mediates many cognitive systems, including those disturbed after chronic exposure to uranium i.e., spatial memory, sleep/wake cycle and locomotor activity.”
Helene Bensoussan^{a}, Line Grancolas^{a}, Bernadette Dhieux-Lestaevel^{b}, Olivia Delissen^{b}, Claire-Marie Vacher^{c}, Isabelle Dublineau^{a}, Philippe Voisin^{a}, Patrick Gourmelon^{a}, Mohammed Taouis^{c}, Philippe Lestaevel^{a}

Uranium is known to travel through the blood to virtually every tissue and organ system in the living body through active transport by blood. It will reduce and for solid precipitates in the hard tissues of the body like bone and also cause kidney stones and kidney disease and the precipitates enlarge with time and chronic exposure. Binding with bicarbonate in the body will also compromise the body's ability to neutralize acids, predisposing to gastric ulcers as well as various muscle pains, cramps and spasms. Highly acidic bodies with compromised acid neutralization abilities, such as contamination with compromising uranium ions, will have higher agitation levels and volatility of behavior. Uranium ions in the liver will compromise blood sugar regulation, causing increased cravings for sugars in the diet, leading to diabetes, metabolic syndromes and obesity, as carbohydrate metabolism is compromised. Further, as blood sugar lacks internal regulation, alcohol and drug use is elevated in statistics, as the body struggles to “just feel good for a little while”. Increased cancer rates are observed with uranium exposure as well as reproductive toxic effects with DNA breakage observed. Compromise to the connective tissues of the body, that cover virtually every surface in the entire body, produce autoimmune diseases such as crippling Lupus. This is exactly what we are seeing in population health statistics on the reservations affected. Further, the toxic effects of uranium are greatly enhanced in the presence of calcium ions, which are known to be generated in ISL mining as well as in runoff waters of the Rocky Mountains over old uranium open pit mines. The Rocky Mountains are high reservoir of calcium carbonate, so ISL mining waters containing uranium as they are known to do, will have even more toxic effects in synergy than what would be expected and predicted of each separately.

### REFERENCE

Medical Effects of Internal Contamination with Uranium
Croatian Medical Journal v. 40, n. 1, March 99 Asaf Durakoviæ
Department of Nuclear Medicine, Georgetown University School of Medicine, Washington D.C., USA

“Uranium as a heavy metal is of particular importance as a complex of uranium and bicarbonate ions, which increases the solubility of uranium in serum. This compound is rather insoluble in water due to the complex ion formation between uranium and bicarbonates. This mechanism determines the transport of ultra filterable uranium from the sites of contamination to the tissues and target organs. In blood, the uranium-bicarbonate complex establishes an equilibrium with non-filterable protein bound uranyl ions, with 60% of uranium bicarbonate-formed and 40% protein-formed. In other studies, 74% of uranium in blood was present in the inorganic compartment of plasma, 32% was protein-formed, whereas 20% was associated with red blood cells. Uranyl salt complexes with bicarbonates are less stable than uranous salt complexes. Reduction of uranium in plasma is not probable, while the uranous salts can be reduced in the intracellular environment. Uranous (IV) retention sites are the bone and kidney, whereas uranyl (VI) ions accumulate in the liver and spleen prior to their redistribution in the renal and skeletal system.”

“Each of the uranyl ions are complexed by two phosphate ions on the surface of bone crystals, with simultaneous release of two calcium ions. The uranous ion produces a toxic effect on the living cells by inhibiting the processes of metabolism of carbohydrates by the inhibition enzyme systems. A uranyl ion replacing a magnesium ion binds the ATP molecule to hexokinase. ATP-uranyl-hexokinase complex blocks the release of phosphate to glucose, inhibiting its first step of metabolic utilization with non-metabolized glucose in the extracellular environment. The toxic effects of uranium were shown to be enhanced by the administration of calcium. The effects of uranium on the nervous system have been described as paralysis of the hind legs, blindness, and loss of coordination in rabbits in the terminal phase of intoxication. Most recent studies indicate significantly higher prevalence of malignant diseases in uranium workers, with increased mutations in underground miners and connective tissue disease, including lupus erythematosus. Reproductive toxicity of uranium in a recent Chinese study includes chromosome aberrations in spermatogonia, causing DNA alterations in the spermatocytes and strand breakage in sperm.”

Testing for Uranium not Required—Faults of the Current Regulatory System

It is interesting to note that currently, testing for even elemental forms of uranium is not required by NRC/Nuclear Regulatory Commission in the mining of uranium, either by open pit or ISL/In Situ Leach mining in the mined aquifers. It is also known, that in the process of ISL uranium mining, organified uranium continues to increase in the mining aquifers without being extracted, as organified uranium does not bind with the resin beads in the ion exchange extraction method, which only is able to extract the inorganic forms of uranium.

Hormone Disruptors

Toxic metals also act as xenohormones and hormone disruptors in the living body. Our hormones are all stereoisomers, meaning atoms are arranged differently in 3 dimensional space, and are subject to the toxic effects of xenohormone environmental toxins. Toxic metals have been shown to act as xenohormones, entering into the cellular receptor sites and skewing the hormone biochemical pathways for estrogen, testosterone, progesterone, cortisol, pregnenolone, thyroid, DHEA, insulin and more. Since hormones are key initiators, regulators and intermediary metabolites of virtually every biochemical reaction in the living body, the protection of their integrity is crucial for their actions. Toxic metals, environmental chemicals and industrial chemical wastes can act as “xenohormones”, and Interfere with natural hormones, enzymes, etc., and cause cancer and other severe ill health compromises.

Further, toxic metals are known to be “xenoestrogens”, a hormone mimic of estrogen, the female and growth hormone. Estrogenic toxicity causes cancer, skin lesions, obesity, fertility problems, accelerated aging, liver problems, learning problems, mood disorders, metabolic syndrome, blood sugar irregularities, blood fat irregularities, increase in breast tissue and size in both males and females, smaller or even undeveloped male genitalia and higher anger and anxiety responses to daily life situations. Mineral imbalances caused by high levels of toxic metals themselves, also are known to cause hormone imbalances of insulin, thyroid, testosterone, progesterone, estrogen and cortisol.

Relevant testing and monitoring for endocrine disruptors of xenoestrogens can be done by testing blood for Total Estrogens and Estradiol, as separate assays. not calculated open from another as is commonly done in medical laboratories. The Total Estrogens test is now considered somewhat obsolete, as it is known to pick up other compounds of estrogenic activity. However this is exactly the activity we are looking for. There are three basic classes of estrogens known in the living body:

Estradiol, which composes approximately 80% of estrogens in the body and is the most potent in activity, made by the ovaries and adrenals

Estrone, which is mostly made and stored in body fat, and the most inflammatory and most associated with environmental exposures

Estriol, which is the only non cancer causing form of estrogen, and a breakdown product of the other two, the weakest acting of the estrogens

Setting up a simple math equation using two assayed numbers of Total Estrogens and Estradiol, we can assess a Toxic Body Burden of Estrogen Dominance and Hormone Disruption, as well as monitor the success of detoxification procedures.

$$\frac{Estradiol at 80 \%}{Total estrogens at 100 \%} = \frac{assayed number}{\begin{matrix}Total Estrogens expected and compared \\ with actual assayed number\end{matrix}}$$

### Example: Testing

If a blood sample tests Estradiol at 50 units, then the expected number for Total Estrogens would be calculated to be 62.5 units, based upon the known of estradiol comprising 80% of Total Estrogens in the living body. Often the actual assayed numbers of Total Estrogens are several hundred times larger, and the difference between the expected number of Total Estrogens and the actual assayed number of Total Estrogens are Xenoestrogenic substances, of which toxic metals and their chemical forms are included. Actively detoxing the affected body with specific detox protocols will show a statistical and dramatic drop in Total Estrogens assayed within 30 to 60 days, without affecting the ratios of the other Estrogens in the body. Symptoms of Estrogen Dominance are also dramatically resolved, including cancer activity and PSA numbers in males.

We see those very problems of Estrogen Dominance exemplified in the most toxic areas of the world, and in increasing statistics overall in the world, as environmental pollution moves around the world. All of the toxic metals studied so far, that are common exposures to man, have shown to be “xenoestrogens” The increase in obesity of animals and humans over the last several decades is directly correlated to the increase of environmental toxins that are known to be fat soluble and deposited in body fat, including heavy metals.

### REFERENCE

J Toxicol Environ Health B Crit Rev. 2009 March: 12(3):206-23. doi: 10.1080/10937400902902062.

The Effects of Metals as Endocrine Disruptors.

Iavicoli 11, Fontana L. Bergamaschi A.

“This review reports current knowledge regarding the roles that cadmium (Cd), mercury (Hg), arsenic (As), lead (PB), manganese (Mn), and zinc (Zn) play as endocrine-disrupting chemicals (EDCs). The influence of these metals on the endocrine system, possible mechanisms of action, and consequent health effects were correlated between experimental animals and humans. Analysis of the studies prompted us to identify some critical issues related to this area and showed the need for more rigorous and innovative studies. Consequently, it was recommended that future studies need to: identify the mechanisms of action, because at the present time only a few have been elucidated in this context, the possible presence of hormesis need to be determined, as currently this was reported only for exposure Cd and As; study the possible additive, synergistic, or antagonistic effects on the endocrine system following exposure to a mixture of metals since there is a lack of these studies available, and in general or occupational environments, humans are simultaneously exposed to different classes of xenobiotics, including metals, but also to organic compounds that might also be EDCs; assess the potential adverse effects on the endocrine system of low-level exposures to metals, as most of the information currently available on EDCs originates from studies in which exposure levels were particularly high; and assess the effects on the endocrine and reproductive systems of other metals that are present in the general and occupational environment that have not yet been evaluated.”

PMID: 19466673 [PubMed—indexed for MEDLINE]

Toxic Metals are Also Known to Denature Protein and Negate the Biochemical Activities of Protein Based Enzymes and Hormones, as Well as Cause Effects in Skeletal Muscles.

Protein makes up a full 90% of the dry weight of the living body. Any living body, any species. Protein is an organic compound composed of long chains of amino acids. Each protein has its own distinct combination of amino acids and also its unique three dimensional shape, and it is the shape that gives it its unique biochemical activity, not simply the chemical formula of its amino acid composition. This is the most important concept in protein, hormone and enzyme biochemistry.

Denaturation is a process in which proteins lose their three dimensional structure/shape which is present in their native state, causing them to unwind and deform, by application of some external stress or compound such as a strong acid or base, a concentrated inorganic salt, an organic solvent (e.g., alcohol or chloroform), radiation or heat. If proteins in a living cell are denatured, this results in disruption of cell activity and possibly cell death. Denatured proteins can exhibit a wide range of characteristics, from conformational change and loss of solubility to communal aggregation to form a solid.

Toxic Metal Inorganic Salts Act to Denature Proteins in Much the Same Manner as Acids and Bases.

Toxic metal salts usually contain Hg+2, Pb+2, Ag+1 Tl+1, Cd+2 and other metals with high atomic weights. Since salts are ionic they disrupt salt bridges in proteins. The reaction of a toxic metal salt with a protein usually leads to an insoluble metal protein salt, meaning that it forms a solid and becomes inactive biochemically. A common example that we all understand and that is epidemic in the human and pet animal population today, is that of insulin. Insulin is a three dimensional folded protein that acts also as a hormone, regulating blood sugar but escorting glucose in the blood into the tissues for storage. If the insulin cannot accomplish this process, then the blood sugar rises to dangerous levels and the patient is diagnosed with Diabetes.

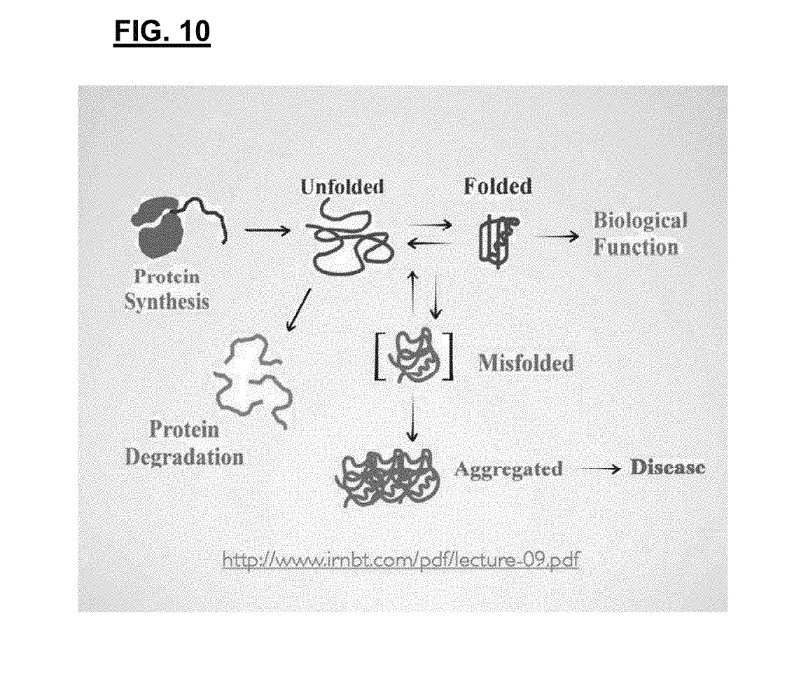

Non-Insulin Dependent Diabetes, or Diabetes Type 2, is the result of such a compromise in the body, with the insulin not able to perform its designated function. It is also called Insulin Resistant Diabetes, because simply giving the affected patient more insulin does not cure the problem. (see FIG. 10—PROTEIN PATHWAYS)

Typical blood testing of insulin reveals the presence of adequate insulin or even higher than normal levels, but conventional blood testing is not capable of viewing the actual three dimensional shape of the molecules to properly asses their actions or lack of. So we typically see the Type 2 diabetic having both high blood glucose along with high insulin levels that are not working effectively. The insulin has been denatured in the blood, and any new insulin that would be still functional when administered to the type 2 diabetic with toxic blood sporting effective levels of some denaturing toxin, will just further deform any new and functional insulin given. Such is the naming of “Insulin Re-Resistance”. The above figures show the production of proteins and their three dimensional folding, and the following figures shows the structure of the insulin molecule.

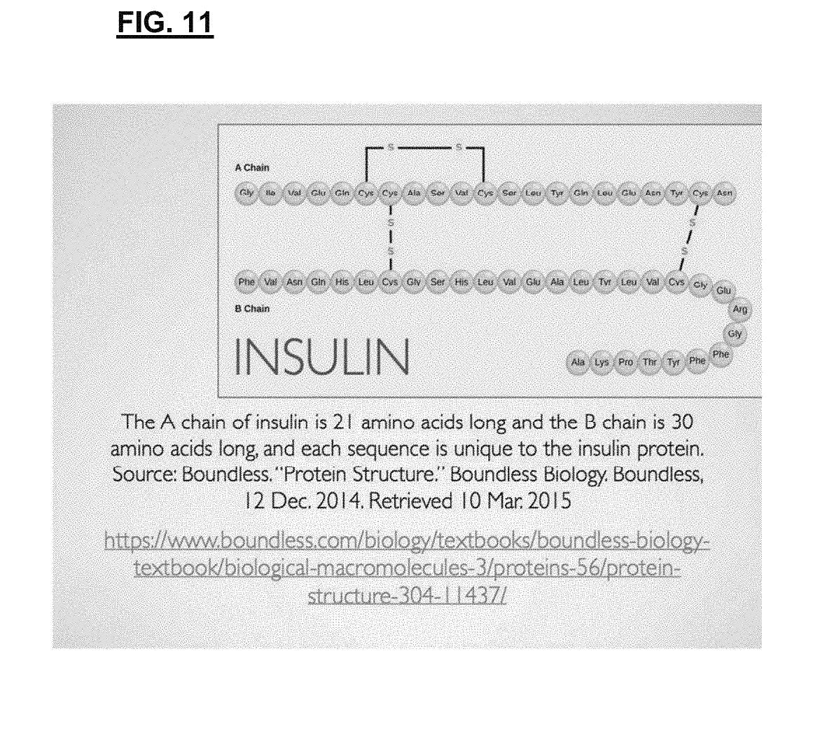

(see FIG. 11—INSUUN CHAIN)

The same scenario is commonly born out with thyroid testing and other natural hormones such as estrogen, testosterone, progesterone, DHEA, cortisol, pregnenolone, etc. We call this scenario in medicine “euthyroid hypothyroid” for thyroid, and appropriately such for the other hormones, where the blood levels show normal levels but the patient manifests hypo hormone symptoms, because the hormones present have been denatured and rendered ineffective. This is a serious problem for medicine today. This is a serious problem in assessing the real toxicity of any toxic metals. Conventional blood testing does not accurately reflect the true health compromise of the sick individual.

Metals cannot be broken down to other elements in Nature or the living body, and in fact, toxin exposure in continuous low levels, formerly thought to be safe, have now been shown to have additive or synergistic effects, where the end effects of a combination of toxin exposure produces more severe health compromises than those that would be expected from each toxin. The common example is that 2+2 now equals 8. Since different chemical forms of minerals and metals can and do exist, and some are more toxic than others, and travel up the food chain at different rates. Different chemical forms of minerals and metals target different organs and tissues of the body. Additionally, each individual toxin is shown to enter the body at levels under the body's detoxification radar of liver detoxification, thus allowing toxic levels of the pollutant to build up over time, until the body becomes so sickened that it cannot help itself anymore in a detox and elimination protective method.

The mostly inorganic forms of arsenic from the mining disaster in the Animas River have not been studied and spectated to accurately assess true toxicity, and you can see there is a huge difference between inorganic species as far as LD50. Organified forms of metals are known to cause higher levels of chronic disease, metabolic compromise and even cancer. They bioaccumulate faster than inorganic forms, and are also known to exhibit genotoxicity as well. Arsenic and other toxic metals can be organified by microorganisms and stream life, to then bioaccumulate at a faster rate in the environment.

Toxicological knowledge on the individual trace element species can lead to more specific legislation of hazardous substances found in animal feed, food, fertilizer, drinking water and even personal care items, as well as environments of fracking, mine waste, ISL mining aquifers, brown fields, rivers, toxic spills, etc. Examples here are arsenic, where the inorganic forms are the most toxic, and mercury, where the organic form methylmercury is more toxic than inorganic mercury. Each toxic metal will be unique.

### Example/Application of the Invention

If arsenic tests at 250 mg/kg×LD50 rate of 14 mg/kg=a toxicity value of 3,500 for total levels of arsenic done as a screening.

If arsenous acid tests at 5 mg/kg×LD50 value of 20 mg/kg=a toxicity value of 100 for this chemical form.

If the sample also contains arsenocholine at 10 mg/kg×LD50 value of 6500=a toxicity value of 65,000.

The sum total of arsenic toxicity is the sum of the new toxicity values, 68,600.

The lower the number, the more toxic the sample tested is as the number is a function of the LD50 numbers.

The same method of calculations can be used for PEL, REL and TLV and other numbers for exposure. Thus by doing this comprehensive study on a contaminated sample of water or soil, a more accurate determination of toxicity to a living organism can be made.

As more evaluation parameters become known and studied, this method of assessing toxicity can be implemented in the future, for example in chronic toxicity and continuous low dose exposure, and with specific tissue targeted organs, as that research become available and even cancer rates for the knowns and unknowns of lead and other toxic metals.

Relevant blood testing and monitoring for endocrine disruptors of xenoestrogens, of which toxic metals are Included, can be done by testing blood for Total Estrogens and Estradiol, as separate assays. not calculated open from another as is commonly done in medical laboratories. The Total Estrogens test is now considered somewhat obsolete, as it is known to pick up other compounds of estrogenic activity. However this is exactly the activity we are looking for. There are three basic classes of estrogens known in the living body:

Estradiol, which composes approximately 80% of estrogens in the body and is the most potent in activity, made by the ovaries and adrenals

Estrone, which is mostly made and stored in body fat, and the most inflammatory and most associated with environmental exposures

Estriol, which is the only form of estrogen not known to cause cancer, and a breakdown product of the other two, the weakest acting of the estrogens

Setting up a simple math equation using two assayed numbers of Total Estrogens and Estradiol, we can assess a Toxic Body Burden of Estrogen Dominance and Hormone Disruption, as well as monitor the success of detoxification procedures.

$$\frac{Estradiol at 80 \%}{Total estrogens at 100 \%} = \frac{assayed number}{\begin{matrix}Total Estrogens expected and compared \\ with actual assayed number\end{matrix}}$$

### Example: Testing

If a blood sample tests Estradiol at 50 units, then the expected number for Total Estrogens would be calculated to be 62.5 units, based upon the known of estradiol comprising 80% of Total Estrogens in the living body. Often the actual assayed numbers of Total Estrogens are several hundred times larger, and the difference between the expected number of Total Estrogens and the actual assayed number of Total Estrogens are Xenoestrogenic substances, of which toxic metals and their chemical forms are included. Actively detoxing the affected body with specific detox protocols will show a statistical and dramatic drop in Total Estrogens assayed within 30 to 60 days, without affecting the ratios of the other Estrogens in the body. Symptoms of Estrogen Dominance are also dramatically resolved, including cancer activity and PSA numbers in males.

I claim:

1. A method for accurately assessing the toxicity of a metal in an environment according to the steps of looking at the quantitative assay numbers of each of the chemical forms in which the metal is known to exist as a function of the different valuations of toxicity, such as LD50 numbers, LD0, LD100, LC50, LOAEL, NOAEL, PEL, REL, TLV etc., for a new terminology of “Toxic Relevance Number” (TRN), with the summary of TRN's of the individual chemical forms shown as TTRN, “Total Toxic Relevance Number”.

2. The method of claim 1 including the step of incorporating the evaluation of toxic metals per the Mineral Wheel, by opposition, biochemical interference of chemical forms, tissue targets, uptake, ultimate destination in the living body, etc., to give a more complete addition of toxicity evaluation of toxic metals in exposure and in the environment.

3. The method of claim 2 including the step of testing and monitoring of the living body for endocrine disruptors of xeno-estrogens can be done by testing blood for Total Estrogens and Estradiol, as separate assays and not calculated open from another as is commonly done in medical laboratories.

4. The method of claim 3 including identifying other compounds of estrogenic activity in which three basic classes of estrogens are known in the living body, the classes comprising
Estradiol, which composes approximately 80% of estrogens in the body and is the most potent in activity, made by the ovaries and adrenals;
Estrone, which is mostly made and stored in body fat, and the most inflammatory and most associated with environmental exposures; and
Estriol, which is the only form of estrogen not known to cause cancer, and a breakdown product of the other two, the weakest acting of the estrogens

5. The method of claim 4 including establishing an equation using two assayed numbers of Total Estrogens and Estradiol, whereby a Toxic Body Burden of Estrogen Dominance and Hormone Disruption is assessed, as well as monitoring the success of detoxification procedures, the equation being:
$$\frac{Estradiol at 80 \%}{Total estrogens at 100 \%} = \frac{assayed number}{\begin{matrix}Total Estrogens expected and compared \\ with actual assayed number\end{matrix}}$$
